(19) United States
(12) Patent Application Publication
(10) Pub. No.: US 2020/0075176 A1
(43) Pub. Date: Mar. 5, 2020

(54) PORTABLE TELEMETRY DEVICE WITH SECONDARY DISPLAY FUNCTION

(71) Applicant: Shenzhen Mindray Bio-Medical Electronics Co., Ltd., Shenzhen (CN)

(72) Inventor: Frank Menzel, Oakland, NJ (US)

(21) Appl. No.: 16/114,552

(22) Filed: Aug. 28, 2018

(51) Int. Cl. G16H 80/00; G06F 3/14; G08B 21/04

(52) U.S. Cl. CPC G16H 80/00; G08B 21/0453; G06F 3/1454

(57) ABSTRACT

A patient-worn, portable telemetry device includes: a processor; a display screen; a graphical interface coupled to the display screen that generates a first graphical display for the display screen; at least one port that receives parameter data from one or more sensors connected to a patient; a first transmitter that wirelessly transmits the parameter data to a remote monitoring station; and a processor that, in response to detecting a predetermined condition, causes a second graphical display generated by the graphical interface to be broadcast to a secondary display device

### TECHNICAL FIELD

The present disclosure relates to monitoring devices for medical telemetry and, more particularly, to portable telemetry devices capable of broadcasting a graphical display to one or more secondary display devices.

### BACKGROUND

Modern medical technology makes extensive use of electronic monitoring of vital signs and other physiological parameters of patients. In some cases, remote monitoring of physiological parameters, or telemetry, is used to allow nurses, doctors, and/or computing devices to determine the health of a patient or detect problems with the patient when the nurse or doctor is not present.

Traditionally, patients have been fitted with various types of sensors including wire leads that are connected to a bedside patient monitor. However, wired connections present challenges when a patient needs to leave the bed or be transferred to another bed or a diagnostic device, such as an X-ray machine. Medical personnel would typically need to disconnect the sensors from the patient monitor, mute any alarms that would ensue, and then reconnect the sensors to the patient monitor after the patient returns to the bed. This is not only time-consuming, but it leaves the patient without monitoring for an extended period.

These problems have been addressed in the art with the use of a portable telemetry device. Sensors are plugged into the portable telemetry device, which is typically worn by the patient using a lanyard around the patient's neck or in a special pocket in the patient's hospital gown. Physiological parameters received from the sensors are broadcast wirelessly to a remote monitoring station. Thus, portable telemetry devices worn by a patient may allow the patient to move about, or be moved between beds or diagnostic devices, without pausing or stopping the gathering of physiological parameters and/or without connecting or reconnecting sensor leads or other cables.

Another advantage of portable telemetry devices is that they are relatively less expensive than bedside patient monitors. As a result, portable telemetry devices have increased in popularity in recent years. Currently, in a typical hospital environment, portable telemetry devices might be used for two-thirds of the patients, while the remaining one-third may be connected to bedside patient monitors or receive no monitoring. One example of a portable telemetry device is the MINDRAY TELEPAK®, certain models of which use proprietary one-way radios operating in very-high frequency (VHF) or ultra-high frequency (UHF) wireless bands to stream patient data over an antenna system to a remote receiver. In the United States, the wireless medical telemetry service (WMTS) provides dedicated protected bands which have been allocated for this purpose.

In recent years, however, there has been a trend toward the use of Wi-Fi (IEEE 802.11) in hospitals because of its ubiquity in computers and other devices. However, Wi-Fi communication frequently experiences disconnection or packet loss as a result of interference by hospital equipment and/or the physical structure of hospitals, making Wi-Fi less reliable than proprietary protocols. To address the unreliability of Wi-Fi, some portable telemetry devices include audible alarms to alert medical personnel if the connection to the remote monitoring station is lost and/or if any of the patient's physiological parameters are outside of predefined limits. However, audible alerts do little to tell an attending nurse, assuming one is in the room, what the specific problem is or how to address it. As such, audible alerts are often missed or ignored.

Many portable telemetry devices have display screens, which can provide alerts and/or parameter information to medical personnel. However, owing to the small size of a portable telemetry device, the display screen is often small, difficult to read, and unable to communicate many details about the patient's condition. The display screen of the portable telemetry device is also small due to limitations imposed by power consumption and available battery life. As such, the display screens of portable telemetry devices are typically turned off until needed.

Furthermore, depending on how the portable telemetry device is worn, it may become stuck under the patient's body or get lost in bedding or the patient's hospital gown, making it difficult to locate the portable telemetry device to check vital signs or alarms. As time is critical in patient care, medical outcomes may suffer as a result of using conventional portable telemetry devices. Audible alerts and display screens on conventional portable telemetry devices do little to mitigate the dangers of Wi-Fi-only connectivity. A patient may go unmonitored and important changes in the patient's condition may go unnoticed by medical personnel.

### BRIEF DESCRIPTION OF THE DRAWINGS

FIG. 1 illustrates one embodiment of a portable telemetry device being used in a clinical setting;

FIG. 2, FIG. 3, and FIG. 4 illustrate a portable telemetry device broadcasting a graphical display to a secondary display device in response to various conditions;

FIG. 5 illustrates the selection of a secondary display device based on proximity;

FIG. 6 illustrates the selection of a secondary display device based on screen size;

FIG. 7 illustrates broadcasting a graphical display to multiple display devices within a particular distance or signaling range;

FIG. 8 is a schematic block diagram of multiple portable telemetry devices in communication with a remote monitoring station;

FIG. 9 is a schematic block diagram showing details of a portable telemetry device and secondary display device; and

FIG. 10 is a flow chart of a method for broadcasting a graphical display from a portable telemetry device to a secondary display device.

### DETAILED DESCRIPTION

The following disclosure solves the aforementioned problems of conventional systems by providing a portable telemetry device capable of broadcasting a graphical display to one or more secondary display devices. One aspect of the disclosure includes a patient-worn, portable telemetry device comprising: a processor; a display screen; a graphical interface coupled to the display screen that generates a first graphical display for the display screen; at least one port that receives parameter data from one or more sensors connected to a patient; a first transmitter that wirelessly transmits the parameter data to a remote monitoring station; and a processor that, in response to detecting a predetermined condition, causes a second graphical display generated by the graphical interface to be broadcast to a secondary display device.

The predetermined condition may include, for example, detecting that at least a subset of the parameter data is outside a predetermined limit, detecting that the first transmitter has lost its connection with the remote monitoring station, and/or detecting that a user has activated a control on the portable telemetry device. The secondary display device may be a television situated near the patient, a tablet computer, a smart phone, a computer monitor, or other similar device.

In one embodiment, the second graphical display is broadcast to a plurality of secondary display devices. For example, graphical display may be broadcast to all secondary display devices within a particular range of the portable telemetry device. The graphical display broadcast to one secondary display device may include one or more different elements from a graphical display broadcast to another secondary display device.

In other embodiments, the secondary display device is selected from a plurality of secondary display devices. For example, the secondary display device having a largest display screen may be selected. Alternatively, the secondary display device closest in proximity to the portable telemetry device may be selected.

The second graphical display may include an alert. Alternatively, or in addition, the second graphical display may include at least a subset of the parameter data received from the one or more sensors. In some embodiments, the first and second graphical displays share one or more common elements. In particular, the second graphical display may mirror the first graphical display. In other embodiments, the second graphical display includes additional information not found in the first graphical display. In some embodiments, a speaker within the portable telemetry device and/or secondary display device generates an audible alert in response to the predetermined condition being detected.

In one embodiment, the graphical display broadcast to the secondary display device is sent via the first transmitter. Alternatively, the second graphical display may be broadcast to the secondary display device via a second transmitter, which may use a different wireless protocol and/or radio than the first wireless transmitter.

The remote monitoring station may include a central monitoring station for a plurality of portable telemetry devices, such as a central nursing station in a hospital or clinic.

In another aspect, a method for patient monitoring includes: receiving parameter data at a patient-worn, portable telemetry device from one or more sensors connected to a patient; generating a first graphical display for a display screen in the portable telemetry device; wirelessly transmitting the parameter data from the portable telemetry device to a remote monitoring station via a first transmitter; and in response to detecting a predetermined condition: generating a second graphical display; and broadcasting the second graphical display from the portable telemetry device to a secondary display device.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

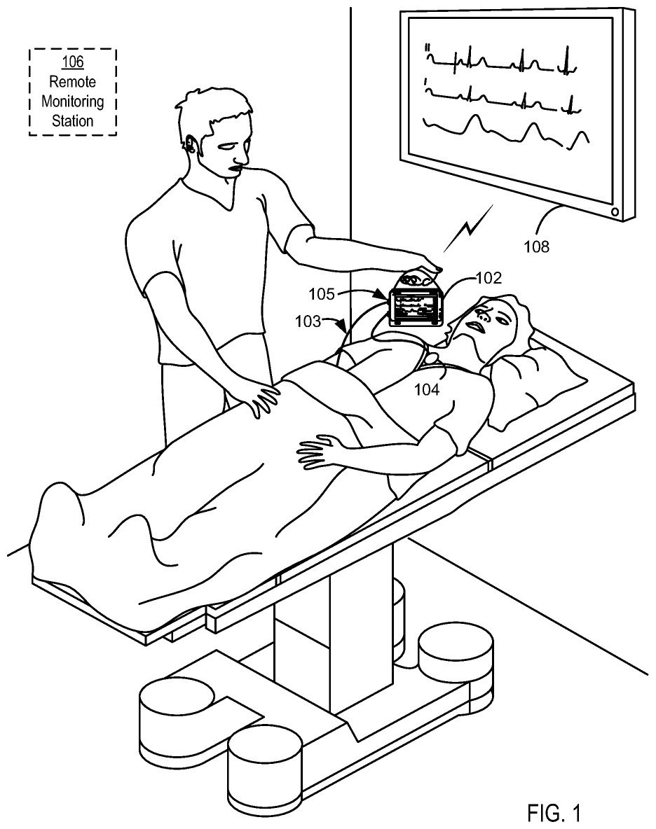

Turning to the figures, FIG. 1 illustrates a portable telemetry device 102 for medical telemetry being used in a clinical setting. In one embodiment, the portable telemetry device 102 is coupled via one or more wires or leads 103 to one or more sensors 104 attached to the patient. For example, the sensors 104 may include one or more electrocardiogram (ECG) sensors, pulse oximetry sensors (e.g., SpO₂), plethysmography sensors, and/or any other sensors capable of detecting physiological parameters of a patient.

The patient may be free to walk or move while wearing the portable telemetry device 102 due to size and capability for wireless communication. As described in greater detail below, the portable telemetry device 102 may include a housing containing a processor, circuitry, computer readable memory, antenna, radios, and/or the like. The form factor of the portable telemetry device 102 may be such that it can be worn by a patient while allowing the patient to move freely.

The portable telemetry device 102 may include one or more ports 105 for coupling the leads 103 to sensors 104 and receiving digital and/or analog signals indicating a physiological condition of a patient. The portable telemetry device 102 may include a human-machine interface (HMI), which may include a display, one or more buttons, and/or indicator lights to allow a human to determine a status of the portable telemetry device 102, enter information, or otherwise interact with the portable telemetry device 102.

The portable telemetry device 102 may be in wireless communication with a remote monitoring station 106 (shown in dashed lines as it may be located in a different room or building), such as a central nursing station in a hospital. The portable telemetry device 102 may transmit physiological data to the remote monitoring station 106 using a unidirectional radio in one embodiment. For example, the portable telemetry device 102 may forward processed or unprocessed sensor data to the remote monitoring station so that a doctor, nurse, or computer can monitor the patient's condition.

In one embodiment, the portable telemetry device 102 may transmit the physiological data as numerical and/or waveform data using a protected frequency via a unidirectional radio. The data may be sent at defined intervals or may be sent every time a buffer is filled with new patient data. In one embodiment, the portable telemetry device 102 may also send an identifier which identifies the patient and/or portable telemetry device 102 to which the physiological data corresponds. Alternatively, or in addition, the portable telemetry device 102 may include a bidirectional radio that can be used for bidirectional communication with the remote monitoring station 106 to receive and/or send control data.

The remote monitoring station 106 may include a computing device, such as a computer, server, or the like. The remote monitoring station 106 may include a processor, circuitry, computer readable memory, antenna, radios, communication ports, and/or the like. In one embodiment, the remote monitoring station 106 may include a computing system for an intensive care ward, step-down ward, or in-patient ward of a hospital.

The remote monitoring station 106 receives the physiological data from the portable telemetry device 102 and stores and/or processes the physiological data. In one embodiment, the remote monitoring station 106 stores the physiological data in memory for later access and/or analysis. The remote monitoring station 106 may process the physiological data to detect problems for the patient, detect whether there is an alarm condition, or perform other analysis. For example, the remote monitoring station 106 may report an alarm condition to a nurse, doctor, or other medical personnel.

The remote monitoring station 106 may also provide control data to the portable telemetry device 102 to configure alarm settings, reset alarms, determine a state or location of the portable telemetry device 102, transfer stored data, or otherwise configure operation of the portable telemetry device 102. In one embodiment, the remote monitoring station 106 may send and receive control data between the portable telemetry device 102 to determine that messages were received or that instructions corresponding to control data were performed.

In one embodiment, the portable telemetry device 102, owing to its small form factor, may include a relatively small display screen, typically having a diagonal measurement of between 1 and 4 inches. As previously discussed, this screen is unable to display much information because of its small size. Moreover, the screen can be easily hidden if the portable telemetry device becomes located beneath the patient or is lost in the patient's bedding or hospital gown.

As described more fully below, the disclosed portable telemetry device 102 is capable of wirelessly broadcasting a graphical display to a secondary display device 108, such as a nearby television set in the patient's room, in response to various conditions. The graphical display sent to the secondary display device may include alerts, parameter data, or the like, which can be more easily seen by a doctor or nurse.

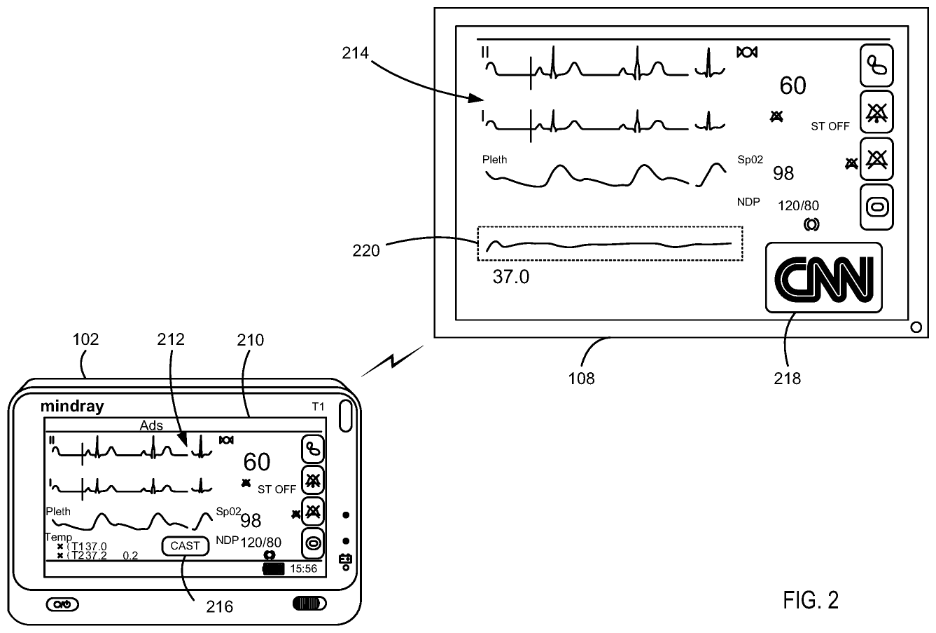

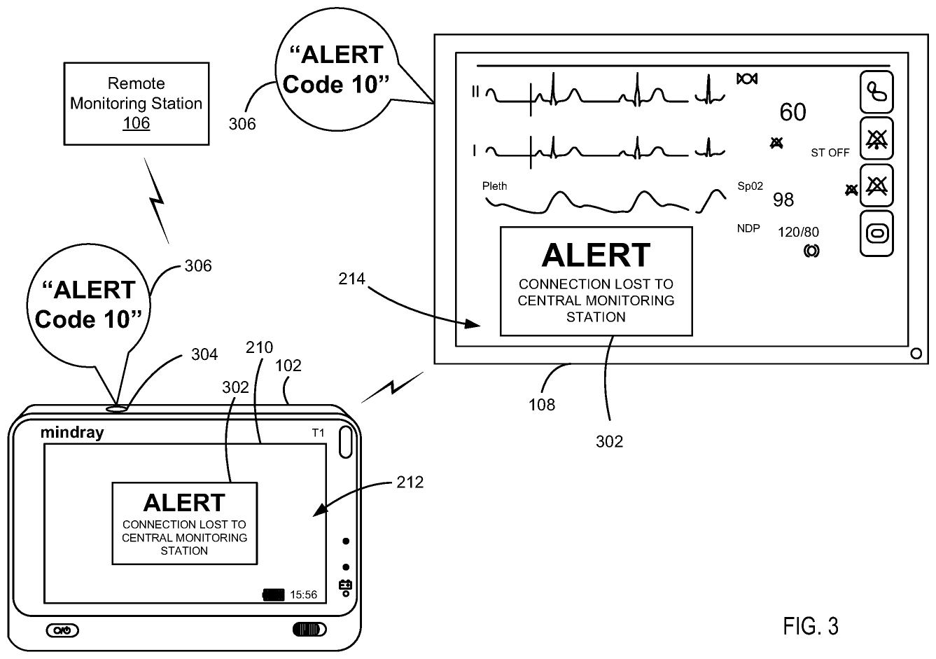

FIG. 2 is an expanded view of a portable telemetry device 102 according to one embodiment. As illustrated, the portable telemetry device 102 includes a display screen 210, which may be touch-sensitive and capable of detecting a user's finger and/or a pointing device, such as a stylus. The display screen 210 may display a first graphical display 212, such as a graphical user interface (GUI), which allows the user to see various alerts and parameter data received by portable telemetry device 102, as well as to interact with portable telemetry device 102 to initiate commands and/or configure the portable telemetry device 102.

The graphical display 212 may include waveforms representing physiological parameters over time, numerical indications of physiological parameters, alerts (i.e., alarms or warnings), as well as virtual controls for activating various features of the portable telemetry device 102.

As noted above, the portable telemetry device 102 may wirelessly broadcast a second graphical display 214 to a secondary display device 108, such as a television located near the patient. The secondary display device 108 could also be a tablet computer or smart phone carried by a doctor or nurse, a computer monitor, or other similar device. The second graphical display 214 may be the same as, or different from, the first graphical display 212.

The graphical display 214 may be wirelessly broadcast in response to detection by the portable telemetry device 102 (and/or the remote monitoring station 106) of a particular condition. For example, in the embodiment shown in FIG. 2, the condition might be the activation of a specific control 216 on the portable telemetry device (real or virtual), such as a “CAST” button.

In response to the portable telemetry device 102 detecting activation of the control 216, the portable telemetry device 102 wirelessly broadcasts the graphical display 214 to the secondary display device 108 for presentation thereon. In one embodiment, the content 218 previously being displayed by the secondary display device 108 may continue be shown in a split screen or picture-in-picture (PIP) arrangement. In other embodiments, the previous content 218 may be entirely replaced by the graphical display 214 sent by the portable telemetry device. In some embodiments, the secondary display device 108 may be triggered to switch on or transition from sleep mode to active operation in response to receiving the broadcast of the graphical display 214.

Technology for wirelessly broadcasting a graphical display from one device to another are known in the art, such as APPLE® AIRPLAY®, as well as SAMSUNG® SMARTVIEW®, QUICKCONNECT®, and SCREEN MIRRORING®. Connection between the portable telemetry device 102 and the secondary display screen 108 may be ad hoc or require previous pairing and/or configuration.

As previously noted, the graphical display 214 on the secondary display device 108 may be the same as, or different from, the graphical display 212 on portable telemetry device 102. For example, in the case of screen mirroring (not shown), the graphical displays 212, 214 may be identical. In other embodiments, as shown in FIG. 2, the second graphical display 214 on the secondary display device 108 may include one or more elements 220 not found in the first graphical display 212 on the portable telemetry device 102. Owing to the typically larger size of the secondary display device 108, room may be available for displaying additional parameter waveforms or trend lines (as shown), additional controls, PIP content 218, or the like. Of course, the second graphical display 214 on the secondary display device 108 may lack one or more elements shown in the first graphical display 212 on the portable telemetry device 102, such as elements that are specific to the operation of the portable telemetry device 102 (e.g., battery level).

FIG. 3 illustrates another condition that may cause the portable telemetry device 102 to broadcast the graphical display 214 to the secondary display device 108. In one embodiment, the portable telemetry device 102 may detect that its connection with the remote monitoring station 106 has been lost. As noted earlier, one of the difficulties with portable telemetry devices 102 relying on certain protocols, such as 802.11, is that the wireless connections may become disconnected due to interference, barriers, or the like. In such cases, the streaming of patient physiological data to the remote monitoring station 106 is interrupted. Where the patient has a large bedside patient monitor with a dedicated screen, such a disconnection can be clearly indicated with a visual and/or audible alert, which will be noticeable to medical personnel. Furthermore, due to the relatively larger display screen of a bedside patient monitor, longer historical trends may be shown, such that when an attending physician or nurse checks on the patient, historical data is more easily observed and any anomalies can be noted, even though the remote monitoring station 106 may not be receiving the parameter data. Thus, medical outcomes are less likely to be jeopardized.

In contrast, a portable telemetry device 102 may, through patient movement or transportation, become situated beneath the patient, concealing its display screen and muffling audible alerts. A doctor or nurse near the patient might not be aware of the disconnection, and even if an audible alert is heard, it may be ignored among the cacophony of sounds in a hospital. A longer interval may go by before the portable telemetry device 102 is physically viewed by medical personal. The relatively small screen may display less historical data, and anomalous readings may be missed by both an attending physician (due to inattention) and a central monitoring station (due to disconnection). Thus, for portable telemetry devices 102 without the features disclosed herein, patient outcomes can be compromised by Wi- Fi interruptions.

In one embodiment, if the portable telemetry device 102 becomes disconnected, it may display a visual alert 302 in its graphical display 212. Alternatively, or in addition, the portable telemetry device 302 may broadcast a graphical display 214 including the visual alert 302 to the secondary display device 108. The visual alert 302 displayed on the secondary display device 108 may be the same as the visual alert 302 displayed on the portable telemetry device 102. Alternatively, the visual alert 302 on the secondary display device 302 may include additional details about the disconnection, e.g., network details, total downtime, etc. In addition to the visual alert 302, patient parameter data may be shown in the graphical display 214 to allow medical personnel to visually monitor the parameter data during the period of disconnection.

A disconnection may be determined in one embodiment by sending packets from the remote monitoring station 106 to the portable telemetry device at regular intervals. If the packets are not received for a time period greater than a predetermined threshold, the connection may be deemed to have been lost. Alternatively, or in addition, the remote monitoring station 106 may detect that physiological data has not been received from the portable telemetry device 102 for a time exceeding the predetermined threshold. In a case of a portable telemetry device 102 have multiple radios, one of which is used for telemetry, the remote monitoring station 106 may use a different radio to notify the portable telemetry device 102 about the disconnection.

In one embodiment, the portable telemetry device 102 may include a speaker 304 to generate an audible alert 306. The audible alert may be a generic sound, such as a beep, to attract the attention of medical personnel. Alternatively, the audible alert 306 may be customized to notify medical personnel concerning the precise nature of the alert 306. For example, the speaker 304 may enunciate a spoken alert 306 that may be coded to avoid worrying the patient (e.g., “code 10” may represent a disconnection alert). In some embodiments, the portable telemetry device 102 may also cause the secondary display device 108 to emit the audible alert 306, which may be more readily heard by medical personnel. The audible alert 306 may be broadcast with the graphical display 214 and output via a speaker (not shown) within the secondary display device 108.

FIG. 4 illustrates a different condition that may cause the portable telemetry device to broadcast a graphical interface to a secondary display device. In some cases, a patient's physiological parameters may be outside predetermined levels (e.g., may exceed or drop below a particular threshold level, and/or leave or enter a particular range). For example, the portable telemetry device 102 (or the remote monitoring station 106) may determine that the patient's resting heart rate exceeds 180 BPM indicating a dangerous level of tachycardia. The portable telemetry device 102 may generate a visual alert 302 and/or audible alert 306 (for presentation on the portable telemetry device 102), optionally identifying the particular physiological parameter that is outside the predetermined level.

In addition, the portable telemetry device 102 may broadcast a graphical display 214 to a secondary display device 108 including a visual alert 302. The alert 302 displayed by the secondary display device 108 may be similar or include one or more different elements from the alert 302 displayed on the portable telemetry device 102. In one embodiment, the graphical display 214 on the secondary display device 108 may include additional information, such as physiological parameter waveforms, numerical parameter data, and the like.

Those skilled in the art will recognize that still other conditions not illustrated in FIGS. 3 and 4 may trigger the broadcast of the graphical display 214 to the secondary display device 214, such as detecting that one or more sensors have become disconnected from the patient, detecting that one or more sensor leads have been pulled from their respective ports, detecting an incorrect connection of the one or more sensor leads, detecting that a battery within the portable telemetry device is low, etc.

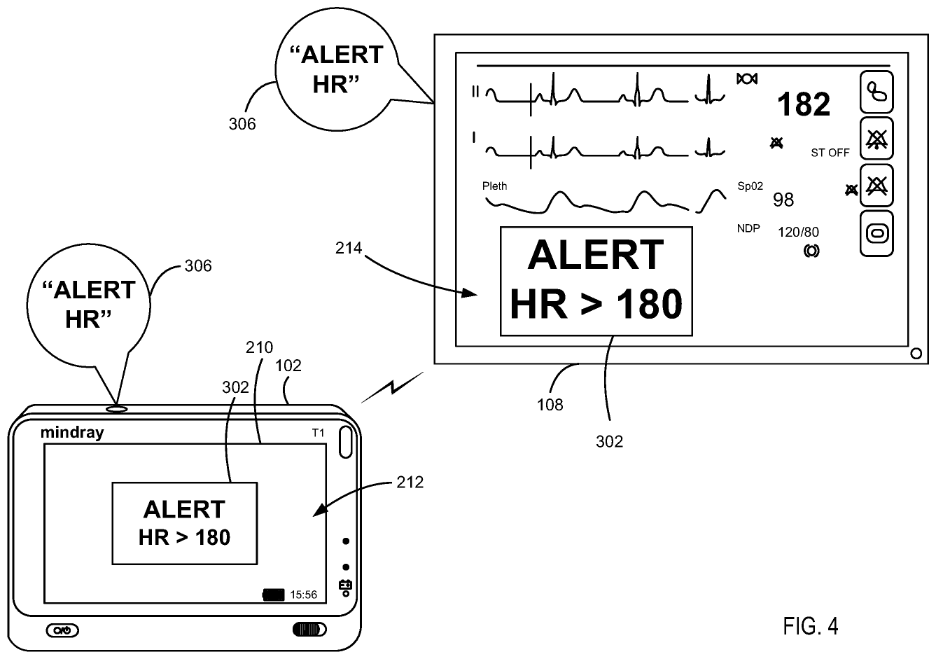

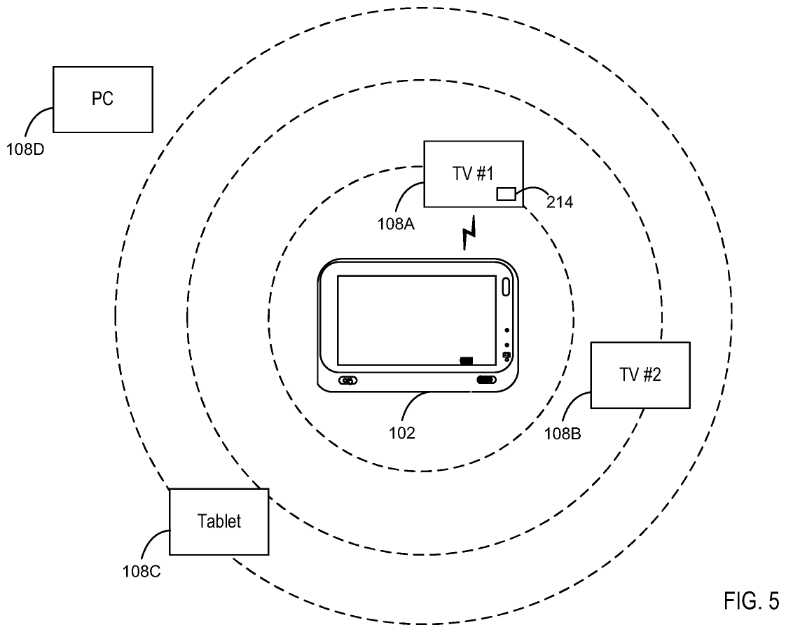

Referring to FIG. 5, the portable telemetry device 102 may select one of a plurality of secondary display devices 108A-D to which to broadcast the second graphical display 214. In some cases, a hospital room may have multiple televisions. Likewise, a room might have various other devices capable of presenting the graphical 214, including a PC, tablet, smart phone, or the like.

In one embodiment, the portable telemetry device 102 selects the secondary display device 108 that is closest in proximity to the portable telemetry device 102. For example, in a room with multiple televisions (TV #1, TV #2), a tablet, and a PC, the portable telemetry device 102 may select TV #1 based on its proximity.

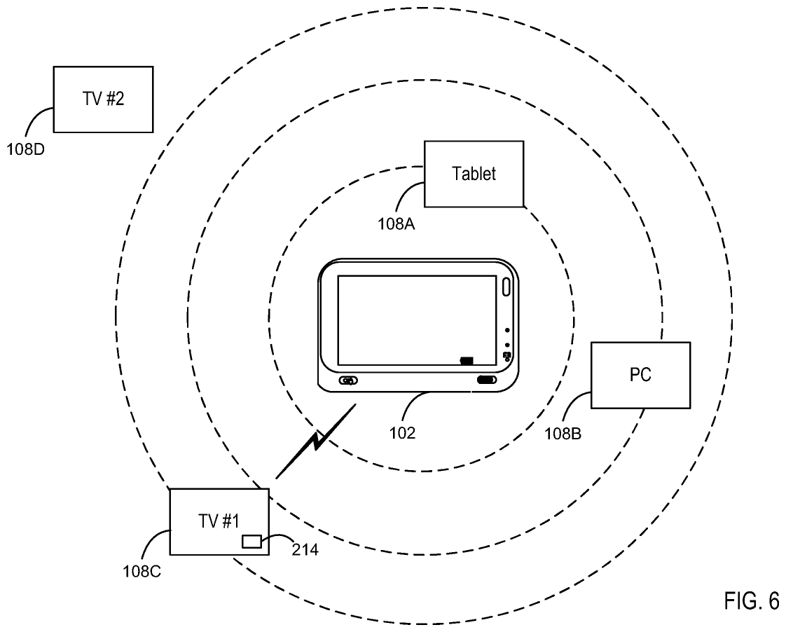

In another embodiment, as shown in FIG. 6, the portable telemetry device 102 selects the secondary display device 108C (TV #1) having the largest display screen. Information about the size of the screen may be obtained, for example, via a handshake protocol between the portable telemetry device 102 and the secondary display device 108. Alternatively, or in addition, screen size information may be specified when the portable telemetry device 102 is configured and/or paired with one or more secondary display devices 108.

As illustrated, despite the fact that a tablet (108A) and PC (108B) are closer in proximity, the portable telemetry device 102 may select TV #1 (108C) based on its relatively larger display screen. TV #2, although having the same or larger size screen, may not be selected in one embodiment based on its relatively greater distance from the portable telemetry device 102. For example, TV #2 may be in another room or beyond the signaling range of the portable telemetry device 102.

Those skilled in the art will recognize that the above-described methods for selecting the secondary display device 108 are only examples and other methods may be used. For example, the portable telemetry device 102 may make a selection based on which secondary device 108 is currently active/available (i.e., not in sleep mode and/or being used by another portable telemetry device 102).

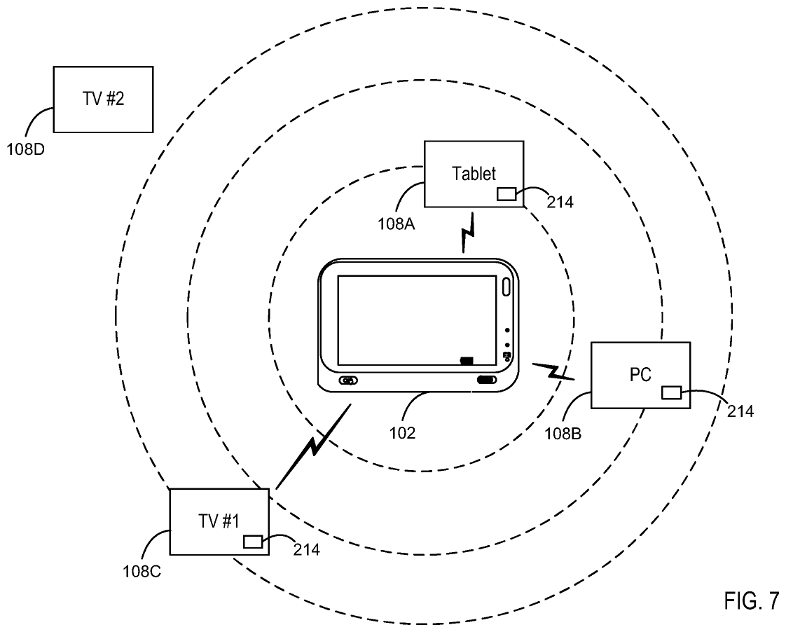

In still another embodiment, as shown in FIG. 7, the portable telemetry device 102 may broadcast the graphical display 214 to a plurality of secondary display devices 108A-C within a predetermined distance or within a signaling distance of the portable telemetry device 102. The graphical interfaces 214 displayed on each respective secondary display device 108A-C may be identical or different and may be based on the relative sizes of the respective secondary display devices 108A-C. For example, TV #1 may be able to display more information than a tablet, such that the portable telemetry device 102 may generate a more detailed graphical display 214 with additional waveforms or other data.

As noted above, one or more secondary display devices 108 may be located in a different room from the portable telemetry device 102 and/or beyond the portable telemetry device's signaling range. In such a case, the determination of which secondary display device 108 is to receive the broadcast may be based on signal strength or whether a particular secondary display device 108 is registered (e.g., during configuration) to be in the same room as the portable telemetry device 102.

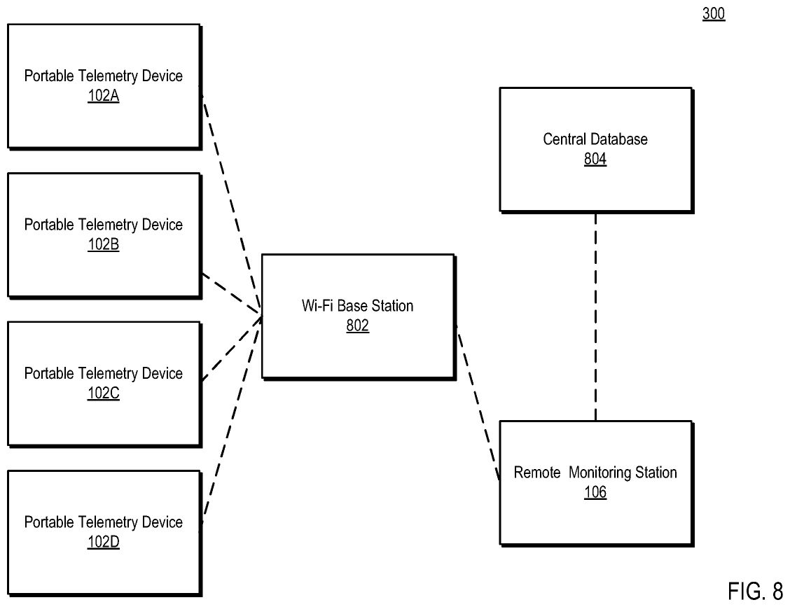

As shown in FIG. 8, a plurality of portable telemetry devices 102A-D may communicate with a remote monitoring station 106, such as a central nursing station of a hospital. The portable telemetry devices 102A-D may transmit patient parameter data directly to the remote monitoring station 106 or via a Wi-Fi base station 802 (other protocols may be used in alternative embodiments). The remote monitoring station 106 may store the patient parameter data in a central database 804 accessible to authorized medical personnel.

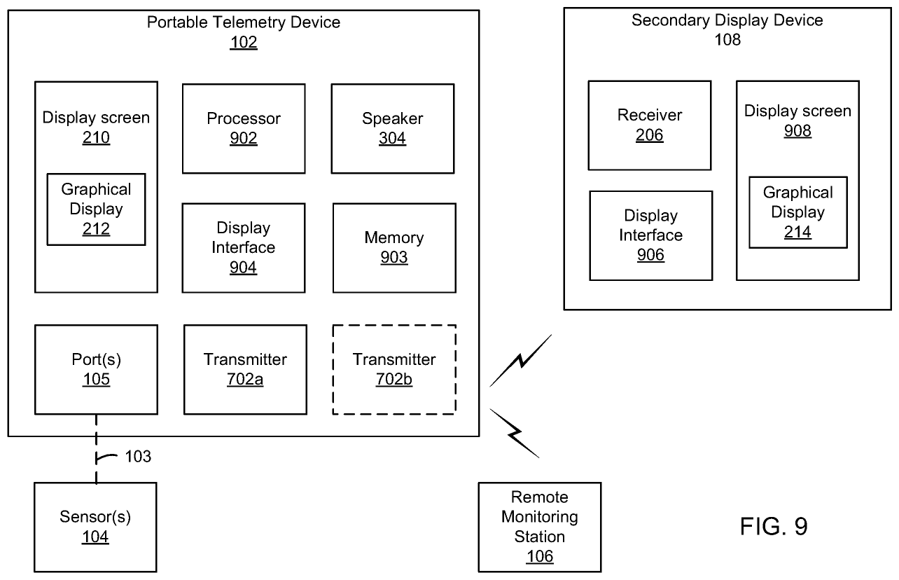

FIG. 9 is a schematic block diagram showing details of a portable telemetry device 102 and a secondary display device 108 according to one embodiment. The portable telemetry device may include a display screen 210, a processor 902, a memory 903, a display interface 904, a speaker 304, one or more transmitters 906A-B, and one or more ports 105, which may be electrically (via wire leads 103) or wirelessly coupled to one or more sensors 104 connected to the patient.

The display screen 210may be embodied as a liquid crystal display (LCD), a light-emitting diode (LED) display, an organic light-emitting diode (OLED) display, or other similar display device. The processor 902 may be a general purpose microprocessor, an application specific processor (ASP), a digital signal processor (DSP), or the like. The memory 903 may include volatile and non-volatile memory using any combination of random access memory (RAM), read-only memory (ROM), electrically erasable programmable read-only memory (EEPROM), magnetic storage, optical storage, or the like.

The display interface 904 may be implemented using general purpose or custom graphics hardware capable of generating a digital or analog signal including the graphical display 212 for presentation on the display screen 210. The port(s) 105 may be implemented using standard connectors for coupling to the sensor(s) 104. In some embodiments, the port(s) 105 may use the Universal Serial Bus (USB) 3.0 standard, although other standards may be used. As used herein, “port” may refer to not only a connector, but any or all circuitry used to convert signals received from the sensors 104 into digital data that can be manipulated by the processor 902. As such, a port 105 may include an analog-to-digital converter (ADC), one or more filters, etc., where a sensor 104 outputs analog signals. Likewise, a port 105 may include hardware for wirelessly receiving parameter data from a suitably equipped sensor 104.

The transmitter(s) 702A-B may include one-way radios (transmit only) operating in very-high frequency (VHF) or ultra-high frequency (UHF) wireless bands to stream parameter data over an antenna to a receiver in the remote monitoring station 104 and/or secondary display device 108. As noted earlier, in the United States, the wireless medical telemetry service (WMTS) provides dedicated protected bands which have been allocated for this purpose and which many hospitals prefer to use over the more widely used industrial, scientific, and medical (ISM) radio bands. Currently the WMTS provides licensed bands in a 608 to 614 megahertz (MHz) range (also known as the 608 MHz band), a 1395 to 1400 MHz range (also known as the 1400 MHz band), and a 1427 to 1432 MHz range. The ISM bands include the popular 2.4 to 2.5 gigahertz (GHz) range and a 5.725 to 5.875 GHz range (also known as the 2.5 GHz and 5 GHz bands) which may be used by routers, wireless home telephones, or the like. Hospitals and other medical providers often use the WMTS bands over the ISM bands because they require less active management and present a smaller patient safety risk. Some portable telemetry devices 102 may use the same protected WMTS bands but have bidirectional radios which allow the device to have additional functionality and act more like a stand-alone patient monitor because they are able to receive, as well as send, data.

In other embodiments, a unidirectional radio may include a VHF/UHF radio in a protected radio band while a bidirectional radio may include an off-the-shelf radio, such as a radio that implements the IEEE 802.11 standard (known to industry groups as Wi-Fi), such as an 802.11 a, b, g, or n. The unidirectional radio may be used for communication of continuous or frequent patient information such as physiological waveforms, measured parameters, and/or alarm information to the remote monitoring station 106. Use of a unidirectional radio that operates in a protected band may provide the dependability that medical teams need for life-critical information. The off-the-shelf radio may be used to listen and respond to commands to the device. For example, these commands may be included in control data to configure operation of the portable telemetry device 102, such as by setting alarm limits, resetting alarms, transferring stored data, etc. The bidirectional radio may also be used to transmit, in one embodiment, the graphical display 214 to the secondary display device 108.

The secondary display device 108 may include a receiver 206, a display interface 906, and a display screen 908 for displaying the graphical display 214 received from the portable telemetry device 102. The receiver 206 may be implemented using a suitable radio technology for receiving a signal from one of the transmitter(s) 702A-B. The display interface 906 may include standard graphical hardware capable of rendering the graphical display 214 on the display screen 908. The secondary display device 108 may further include a speaker (not shown) to generate audible alerts 306 shown in FIGS. 3 and 4.

In one embodiment, the secondary display device 108 might not have dedicated hardware for receiving a wireless transmission from the portable telemetry device 102. In such a case, the secondary display device 108 may rely on a commercially-available adapter/dongle (not shown), such as a GOOGLE® CHROMECAST® or the like, which is plugged into a video input, such as a High-Definition Multimedia Interface (HDMI) input, of the secondary display device 108.

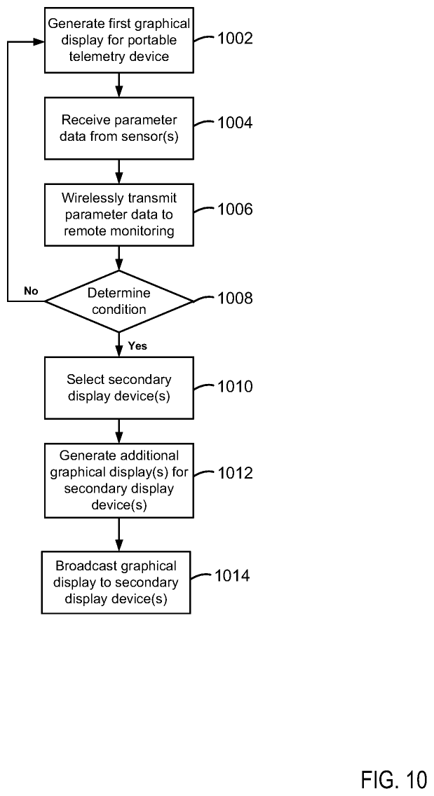

FIG. 10 a flowchart of a method for broadcasting a graphical display from a portable telemetry device to a secondary display device. The portable telemetry device may generate 1002 a first graphical display for an integrated display screen. The graphical display may include parameter data in the form of numerical values, trend lines, and/or waveforms, as well as various alerts, such as alarms or warnings, as described above. The portable telemetry device may receive 1004 patient parameter data from one or more sensors, after which the portable telemetry device may wirelessly transmit 1006 the received parameter data to a remote monitoring station, such as a central nursing station.

The portable telemetry device may determine 1008 whether a particular condition has occurred. For example, as depicted in FIG. 2, the particular condition may be that the user has pressed a control on the portable telemetry device, such as a “CAST” button. Alternatively, as illustrated in FIG. 3, the condition may be that the portable telemetry device has lost its connection with the remote monitoring station. In still other embodiments, as shown in FIG. 4, the condition may be that one or more patient parameters are outside predetermined levels.

If one of the particular conditions has not occurred, control returns to the step 1002. However, in response to determining that one of the particular conditions has occurred, the portable display device 102 may select 1010 one or more secondary display devices(s) to which to broadcast a second graphical display. As illustrated in FIG. 5, the one or more secondary display device(s) may be selected based on proximity. Alternatively, as shown in FIG. 6, the one or more secondary display device(s) may be selected based on screen size. As depicted in FIG. 7, multiple secondary display devices may be selected within a particular range of the portable telemetry device.

Thereafter, or at the same time, the portable display device 102 may generate 1012 one or more additional graphical display(s) for the one or more selected secondary display device(s). The additional graphical display(s) are then broadcast 1014 to the secondary display device(s) for presentation thereon.

As will be appreciated by one of ordinary skill in the art, principles of the present disclosure may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element.

What is claimed is:

1. A patient-worn, portable telemetry device comprising:
a processor;
a display screen;
a graphical interface coupled to the display screen that generates a first graphical display for the display screen;
at least one port that receives parameter data from one or more sensors connected to a patient;
a first transmitter that wirelessly transmits the parameter data to a remote monitoring station; and
a processor that, in response to detecting a predetermined condition, causes a second graphical display generated by the graphical interface to be broadcast to a secondary display device.

2. The portable telemetry device of claim 1, wherein the predetermined condition comprises detecting that at least a subset of the parameter data is outside a predetermined limit.

3. The portable telemetry device of claim 1, wherein the predetermined condition comprises detecting that the first transmitter has lost a connection with the remote monitoring station.

4. The portable telemetry device of claim 1, wherein the predetermined condition comprises detecting that a user has activated a control on the portable telemetry device.

5. The portable telemetry device of claim 1, wherein the secondary display device comprises a television near the patient.

6. The portable telemetry device of claim 1, wherein the processor causes the second graphical display to be broadcast to a plurality of secondary display devices.

7. The portable telemetry device of claim 1, wherein the processor causes the second graphical display to be broadcast to all secondary display devices with a particular range of the portable telemetry device.

8. The portable telemetry device of claim 1, wherein the processor selects the secondary display device from a plurality of secondary display devices.

9. The portable telemetry device of claim 8, wherein the processor selects the secondary display device of the plurality of the plurality of secondary display devices having a largest display screen.

10. The portable telemetry device of claim 8, wherein the processor selects the secondary display device of the plurality of the plurality of secondary display devices that is closest to the portable telemetry device.

11. The portable telemetry device of claim 1, wherein the second graphical display comprises an alert.

12. The portable telemetry device of claim 1, wherein the second graphical display comprises at least a subset of the parameter data.

13. The portable telemetry device of claim 1, wherein the first and second graphical displays share one or more common elements.

14. The portable telemetry device of claim 1, wherein the second graphical display mirrors the first graphical display.

15. The portable telemetry device of claim 1, wherein the second graphical display includes additional information not found in the first graphical display.

16. The portable telemetry device of claim 6, wherein the graphical display broadcast to a first secondary display device includes one or more different elements from the graphical display broadcast to a second secondary display device.

17. The portable telemetry device of claim 1, further comprising:
a speaker that generates an audible alert in response to the predetermined condition being detected.

18. The portable telemetry device of claim 1, wherein the second graphical display is broadcast via the first transmitter.

19. The portable telemetry device of claim 1, further comprising:
a second transmitter;
wherein second graphical display is broadcast via the second transmitter.

20. (canceled)

21. A method for patient monitoring, comprising:
receiving parameter data at a patient-worn, portable telemetry device from one or more sensors connected to a patient;
generating a first graphical display for a display screen in the portable telemetry device;
wirelessly transmitting the parameter data from the portable telemetry device to a remote monitoring station via a first transmitter; and
in response to detecting a predetermined condition:
generating a second graphical display; and
broadcasting the second graphical display from the portable telemetry device to a secondary display device.

22-40. (canceled)
